(19) United States
(12) Patent Application Publication
(10) Pub. No.: US 2022/0233060 A1
(43) Pub. Date: Jul. 28, 2022

(54) Device for endoscopic foreign-body removal

(71) Applicant: Miguel Ramos, Ocala, FL (US)

(72) Inventor: Miguel Ramos, Ocala, FL (US)

(21) Appl. No.: 17/648,552

(22) Filed: Jan. 21, 2022

Related U.S. Application Data
(60) Provisional application No. 63/140,289, filed on Jan. 22, 2021.

(51) Int. Cl. A61B 1/00; A61B 1/015; A61B 1/273

(52) U.S. Cl. CPC A61B 1/00137; A61B 1/2733; A61B 1/015; A61B 1/00128

(57) ABSTRACT

The endoscope adapter for obstruction removal attaches to an endoscope via a flexible coupler/collar. The endoscope uses a pump to create suction, the low pressure causing a flow through the adapter. A constriction, or venturi, between the endoscope tip and adapter creates an increase in suction pressure, resulting in an increased effectiveness in gripping and removing obstructions, such as partially-chewed food. The endoscope adapter for obstruction removal includes two primary features: First, the tip is a larger diameter than the coupler, thus increasing the area for gripping the food or bolus. Second, a construction, forming a venturi, between the location where suction is applied by the endoscope, increasing the resulting suction.

### CROSS-REFERENCE TO RELATED APPLICATION

This application claims priority to U.S. Prov. App. Ser. No. 63/140,289 filed Jan. 22, 2021, titled Device for endoscopic foreign-body removal.

### FIELD

This invention relates to the field of gastroenterology and more particularly to a device to aid in the removal of foreign bodies from the esophagus.

### BACKGROUND

Generally, humans chew their food in their mouth, where the food is broken down and mixed with saliva, after which it is swallowed and passes into the stomach.

While this process generally works, at times the partially-chewed food, or bolus, stops short of the stomach. Or an object, swallowed by accident or by a child, becomes trapped in a narrow portion in the esophagus. In either situation, a foreign body is lodged in the esophagus and medical intervention is required for removal.

Currently, removal of such foreign bodies involves the use of non-ideal devices, such as snares, forceps, or baskets affixed to the end of a commercially-available endoscope. These devices have significant drawbacks, often causing food to separate into individual pieces. This lengthens the procedure and causes unneeded trauma to the patient.

What is needed is a device to effectively aid in the removal of obstructions from the esophagus, specifically food.

### SUMMARY

The endoscope adapter for obstruction removal attaches to an endoscope via a flexible coupler/collar. The endoscope uses a pump to create suction, the low pressure causing a flow through the adapter. A constriction, or venturi, between the endoscope tip and adapter creates an increase in suction pressure, resulting in an increased effectiveness in gripping and removing obstructions, such as partially-chewed food.

The endoscope adapter for obstruction removal includes two primary features:

First, the tip is a larger diameter than the coupler, thus increasing the area for gripping the food or bolus.

Second, a construction, forming a venturi, is placed between the location where suction is applied by the endoscope and the tip of the adapter, increasing the resulting suction.

The preferred embodiment of the endoscope adapter for obstruction removal includes a tip with a 12 mm internal diameter, into which the food or obstruction is drawn. This diameter reduces to a 2 mm internal diameter for a length of 2 mm, creating a venturi, or restriction. Internal diameter then increases to 10 mm at the flexible collar that affixes to the distal tip of the endoscope. The suction inlet at the distal tip of the endoscope measures 2.5 mm.

In total the preferred embodiment is a length of 28 to 30 mm.

In the preferred embodiment the flexible coupler has a diameter of 8.5 millimeters. With the preferred embodiment having an internal diameter of 12 millimeters, the result is that the body of the preferred embodiment has a 40% larger diameter than the coupling diameter.

In the second embodiment the internal diameter is expanded to 15 millimeters.

With the second embodiment having an internal diameter of 15 millimeters, the result is that the body of the second embodiment has an approximately 75% larger diameter than the coupling diameter.

The body of the device having larger internal diameter than the coupling improves its ability to remove larger objects.

The closest existing device is a device adapted from use as a banding device. It is a cylindrical shape and attaches to the distal tip of an endoscope using a flexible collar. The device is 25 mm in length with an opening diameter of 10 mm.

The narrowing of the internal diameter to create the venturi has the potential of obstructing the view of the camera, or optical fibers, present in the distal tip of the endoscope. To address this, the material used around the venturi is clear, permitting light to pass through without distortion. This allows the user to see through the endoscope adapter for obstruction removal.

The purpose of an endoscopy is to view the gastrointestinal tract. This may include the upper gastrointestinal tract, the stomach, and the lower GI tract. In order to enhance the user's view, irrigation and suction are available to clear any obstructive debris. The endoscope adapter for obstruction removal allows flow both to and from the endoscope distal tip, thus allowing water to exit the endoscope adapter into the body, as well as for suction to draw items into the endoscope adapter. The user may also use the irrigation feature of the endoscope to expel water, which can clean the device and help the user to better view the esophagus and the obstruction.

The suction pumps generally used in conjunction with an endoscope can create a maximum suction of 450 mm Hg.

Discussion will now focus on the preferred embodiment.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a first isometric view of the endoscope adapter for obstruction removal.

FIG. 2 illustrates a view of a typical flexible endoscope.

FIG. 3 illustrates a side view of the endoscope adapter for obstruction removal.

FIG. 4 illustrates a cross-sectional view of the endoscope adapter for obstruction removal.

FIG. 5 illustrates an isometric cross-sectional view of the endoscope adapter for obstruction removal.

FIG. 6 illustrates an isometric end view of the tip of the endoscope adapter for obstruction removal.

FIG. 7 illustrates a view of the tip of the endoscope adapter for obstruction removal.

FIG. 8 illustrates an isometric end view of the base of the endoscope adapter for obstruction removal.

FIG. 9 illustrates a view of the base of the endoscope adapter for obstruction removal.

FIG. 10 illustrates a side view of a second embodiment of the endoscope adapter for obstruction removal.

FIG. 11 illustrates a cross-sectional view of a second embodiment of the endoscope adapter for obstruction removal.

FIG. 12 illustrates an isometric cross-sectional view of a second embodiment of the endoscope adapter for obstruction removal.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

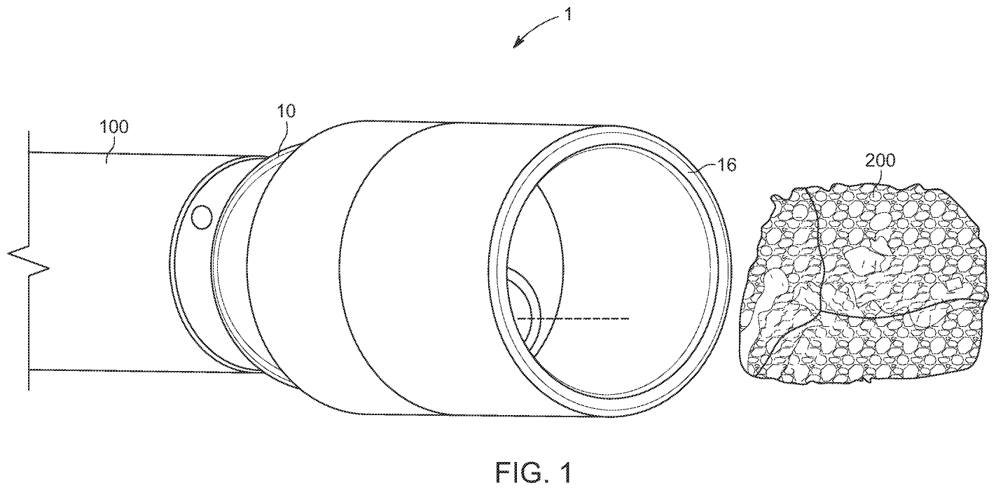

Referring to FIG. 1, a first isometric view of the endoscope adapter for obstruction removal is shown.

The endoscope adapter for obstruction removal 1 is shown, with the flexible coupler 10 ready to affix to the flexible endoscope 100.

An obstruction 200, such as food or bolus, is shown ready to be drawn into the tip 16 of the endoscope adapter for obstruction removal 1.

The first embodiment of the endoscope adapter for obstruction removal one is shown with an exterior diameter of 12 millimeters. This embodiment is intended for use in patients with smaller esophagus, for example, children.

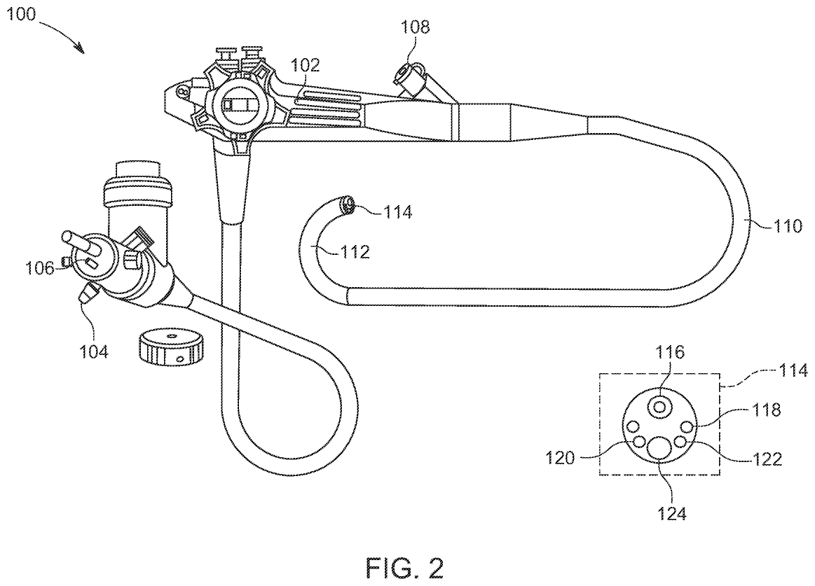

Referring to FIG. 2, a view of a typical flexible endoscope is shown.

The flexible endoscope 100 includes a body 102, suction pump connection 104, air pump connection 106, instrument inlet 108, insertion tube 110, bending section 112, and distal tip 114.

Focusing on a close-up view of the distal tip 114, also shown is a lens 116, light guide 118, irrigation nozzle 120, insufflation nozzle 122 (for injection of air), and instrument and suction channel 124.

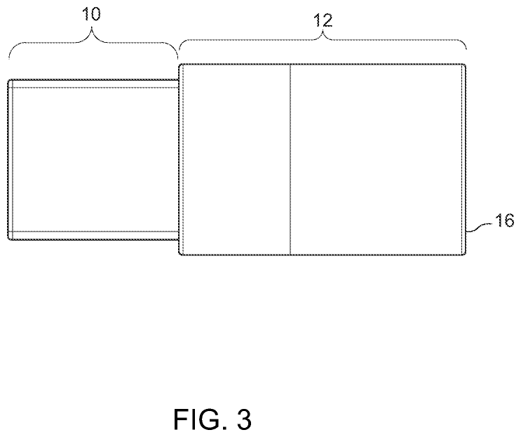

Referring to FIG. 3, a side view of the endoscope adapter for obstruction removal is shown.

The endoscope adapter for obstruction removal 1 includes primary components flexible coupler 10, body 12, and tip 16.

The flexible coupler 10 is preferable made from a pliable material, such as silicone.

The body 12 is preferably made from a material that is stiffer than the flexible coupler 10, such as Polyethylene, Polypropylene, or similar.

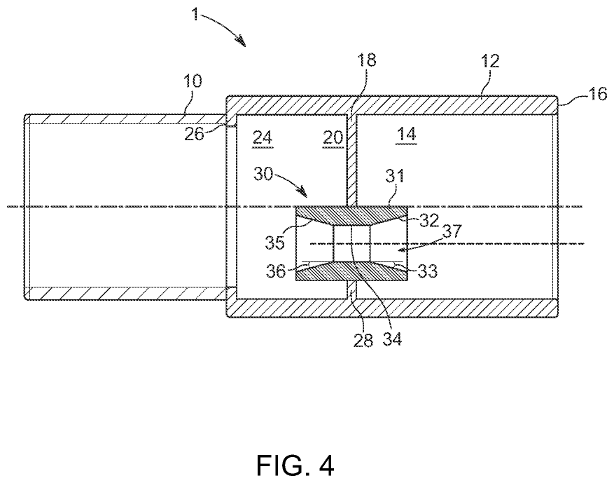

Referring to FIG. 4, a cross-sectional view of the endoscope adapter for obstruction removal is shown.

Note that the diameter of the body 12 is greater than the diameter of the flexible coupler 10.

Viewing the internals of the endoscope adapter for obstruction removal 1, primary parts again include flexible coupler 10, body 12, and tip 16.

Within the tip 16 is the obstruction chamber 14, into which the food, bolus, or other object is drawn.

The viewing window 18 sits within a viewing recess 20 within the interior wall 28. The viewing window 18 allows light to pass through the endoscope adapter for obstruction removal 1 and into the light guide 118 (see FIG. 2), thus allowing the user to guide the distal tip 114 (see FIG. 2). The thickness of the viewing window 18 is optionally reduced to minimize distortion.

The venturi 30 sits within the interior wall 28. The venturi 30 includes a venturi body 31.

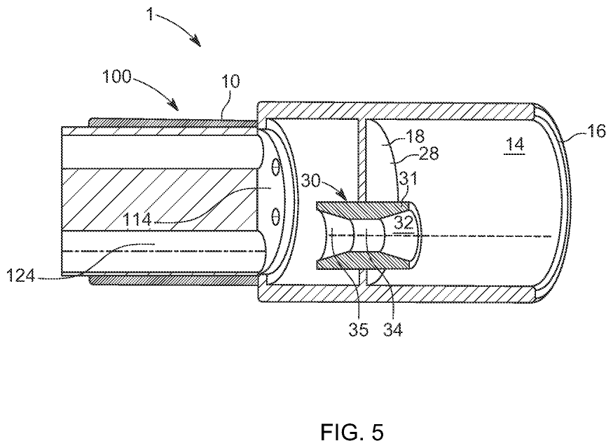

The venturi 30 is preferably placed below the centerline of the body 12. By placing the venturi below the center, the viewing window is increased in size to provide the user a clear and unobstructed view of the obstruction chamber 14. The venturi is best located in-line with, or biased toward, the instrument and suction channel 124 (see FIG. 5) of the flexible endoscope 100 (see FIG. 5).

The venturi 30 is divided into a venturi converging section 32 and venturi diverging section 35 separated by the venturi throat 34.

The venturi converging section 32 has walls set at venturi converging angle 33.

The venturi diverging section 35 has walls set at venturi diverging angle 36.

Venturi converging angle 33 and venturi diverging angle 36 measured with respect to the venturi throat 34 are each preferably equals to or less than 15 degrees.

The venturi 30 allows flow through the endoscope adapter for obstruction removal 1 in direction of flow 37, connecting the interior chamber 24 to the obstruction chamber 14. Restated, the venturi 30 is the fluid communication pathway between the endoscope and the obstruction chamber 14.

The distal tip 114 (see FIG. 2) of the flexible endoscope (see FIG. 2) is inserted into the flexible coupler 10, resting against the endoscope stop 26 of the interior chamber 24, the endoscope stop 26 preventing over-insertion.

The interior chamber 24 is also referred to as the first chamber, and the obstruction chamber 14 referred to as the second chamber.

Referring to FIG. 5, an isometric cross-sectional view of the endoscope adapter for obstruction removal is shown.

With the distal tip 114 inserted into the coupling 10, the arrangement of the endoscope adapter for obstruction removal 1 with respect to the distal tip 114 is visible.

The venturi 30, venturi body 31, venturi converging section 32, venturi throat 34, and venturi diverging section 35 are again visible.

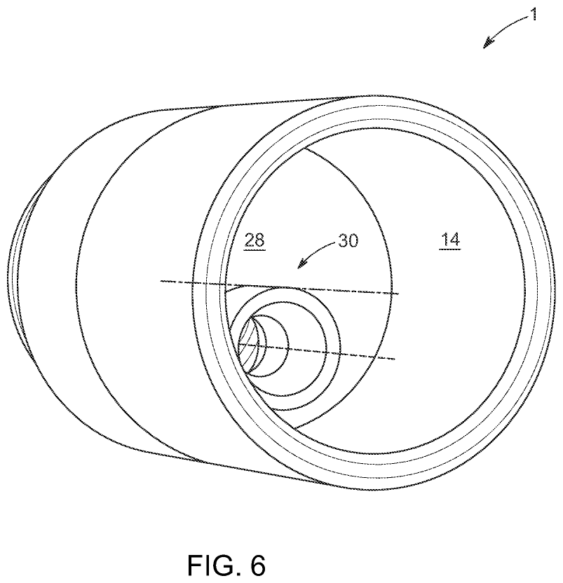

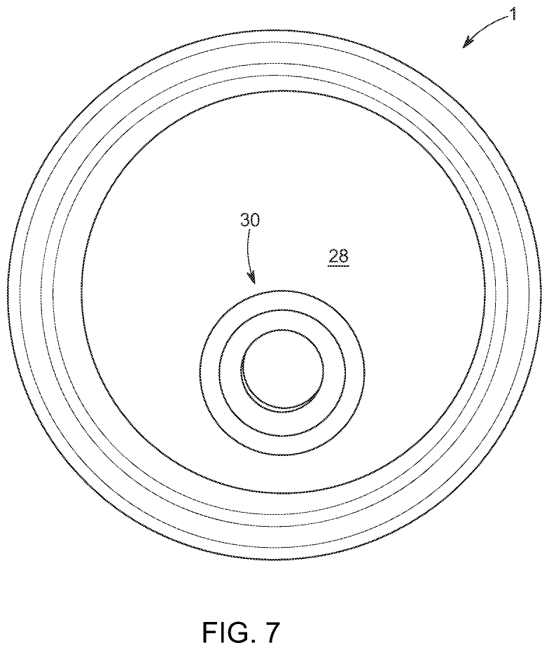

Referring to FIGS. 6 and 7, an isometric end view, and an end-on view, of the tip of the endoscope adapter for obstruction removal are shown.

The endoscope adapter for obstruction removal 1 is shown with obstruction chamber 14, and the venturi 30 protruding through the interior wall 28.

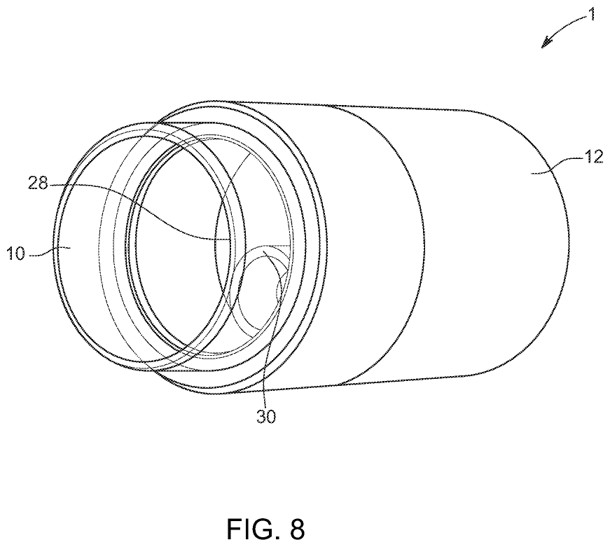

Referring to FIG. 8, an isometric end view of the base of the endoscope adapter for obstruction removal is shown.

The endoscope adapter for obstruction removal 1 is shown with flexible coupler 10, the venturi 30 supported by the interior wall 28.

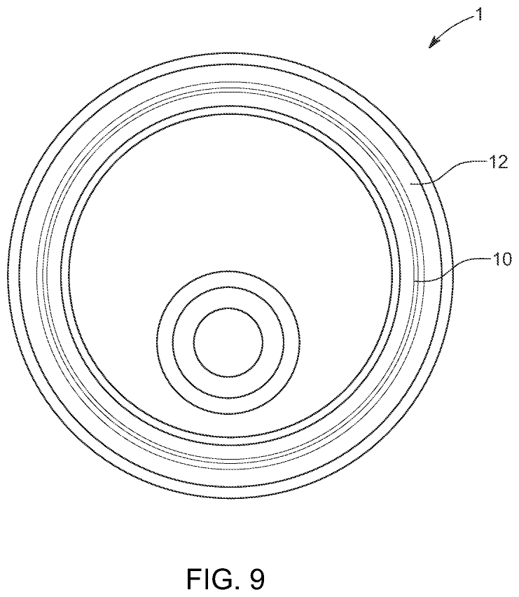

Referring to FIG. 9, a view of the base of the endoscope adapter for obstruction removal is shown.

The endoscope adapter for obstruction removal 1 is shown with body 12 and flexible coupler 10.

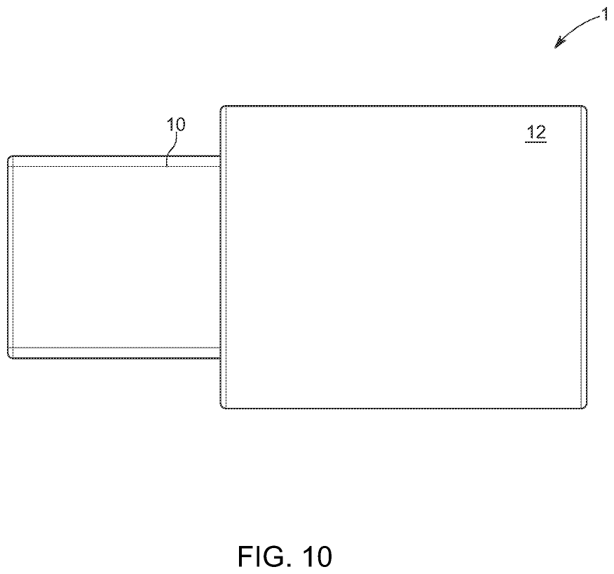

Referring to FIG. 10, a side view of a second embodiment of the endoscope adapter for obstruction removal is shown.

The second embodiment of the endoscope adapter for obstruction removal 1 has an exterior diameter of 15 millimeters. This second embodiment is designed for use in patients with larger esophagus, for example adults.

Note that the diameter of the body 12 is again larger than the flexible couple of 10.

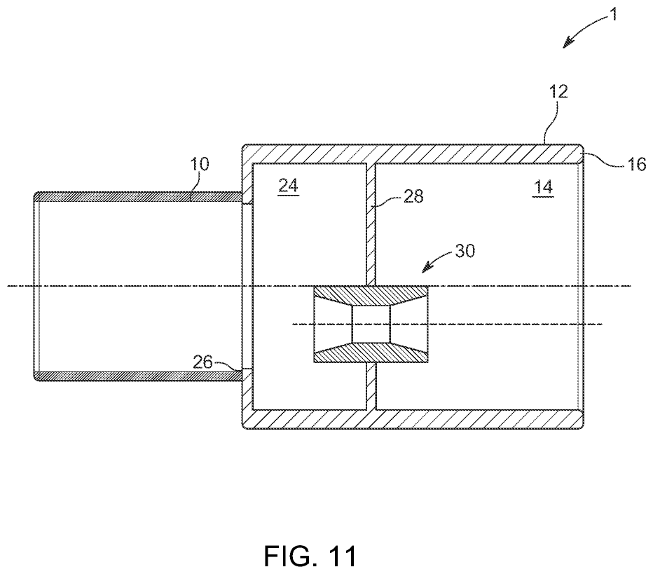

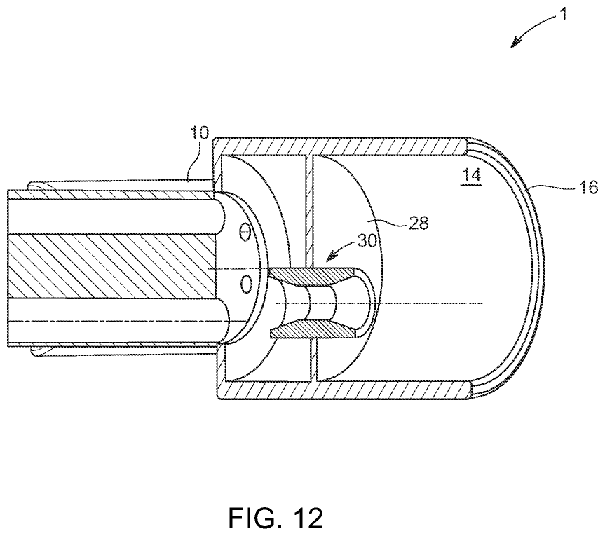

Referring to FIGS. 11 and 12, a cross-sectional view and isometric cross-sectional view of a second embodiment of the endoscope adapter for obstruction removal are shown.

The endoscope adapter for obstruction removal is shown with flexible coupler 10, body 12, interior chamber 24, endoscope stop 26, interior wall 28, venturi 30, obstruction chamber 14, and tip 16.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A device for removing an obstruction from an esophagus of a patient, the device comprising:
a body;
the body having a first chamber and a second chamber;
the first chamber and the second chamber divided by an interior wall;
the first chamber adjoining an endoscope when in use;
the second chamber to capture the obstruction;
a venturi;
the venturi including a converging section, a diverging section, and a throat;
the venturi connecting the first chamber and the second chamber;
the venturi supported by the interior wall;
whereby the device uses a pressure change of the venturi to improve on a suction force created by the endoscope.

2. The device for removing an obstruction from an esophagus of a patient of claim 1, wherein:
the first chamber has a first diameter and the second chamber has a second diameter; and
this first diameter is equal to the second diameter.

3. The device for removing an obstruction from an esophagus of a patient of claim 1, further comprising:
a flexible coupler;
the flexible coupler adjacent to the first chamber;
the flexible coupler allowing the device to be placed on and removed from a tip of the endoscope.

4. The device for removing an obstruction from an esophagus of a patient of claim 3, wherein:
the first chamber has a first diameter and the second chamber has a second diameter; and
this first diameter is equal to the second diameter.

5. The device for removing an obstruction from an esophagus of a patient of claim 1, wherein:
the converging section includes a venturi converging angle with respect to the throat;
the venturi converging angle being less than or equal to 15 degrees.

6. The device for removing an obstruction from an esophagus of a patient of claim 1, wherein:
the venturi is centered below a center of the body, creating a viewing recess through the interior wall;
the viewing recess allowing a user to have an unobstructed view through to the second chamber via a camera associated with the endoscope.

7. A device for attachment to an endoscope, the endoscope including a suction line and a camera, the device to aid in removal of an obstruction from an esophagus, the device comprising:
an obstruction chamber;
an interior chamber;
the interior chamber and the obstruction chamber divided by an interior wall;
a venturi;
the venturi is the path for fluids passing from the obstruction chamber to the interior chamber;
the venturi supported by the interior wall;
whereby the venturi improves performance of the endoscope, aiding in the removal of the obstruction from an esophagus.

8. The device of claim 7, wherein:
the interior chamber has a first diameter and the obstruction chamber has a second diameter; and
this first diameter is equal to the second diameter.

9. The device of claim 7, further comprising:
a flexible coupler;
the flexible coupler adjacent to the interior chamber;
the flexible coupler allowing the device to be placed on and removed from a tip of the endoscope.

10. The device of claim 9, wherein:
the interior chamber has a first diameter and the obstruction chamber has a second diameter;
the flexible coupler has a coupler interior diameter;
this first diameter is equal to the second diameter;
the coupler interior diameter is less than the first diameter;
whereby the obstruction chamber is able to hold an obstruction larger in diameter than the coupler interior diameter.

11. The device of claim 7, wherein:
the venturi includes a converging section, a diverging section, and a throat;
the converging section includes a venturi converging angle with respect to the throat;
the venturi converging angle being less than or equal to 15 degrees.

12. The device of claim 7, wherein:
the venturi is placed below a center of the interior chamber, creating a viewing recess through the interior wall;
the viewing recess allowing a user to have an unobstructed view through to the obstruction chamber via a camera associated with the endoscope.

13. A device for removing food from an esophagus, the device comprising:
a body;
the body including a flexible coupler and a tip;
the flexible coupler to connect to a flexible endoscope;
the tip to collect food;
a fluid communication pathway connecting the flexible coupler to the tip;
a restriction within the fluid communication pathway, creating a venturi;
whereby the flexible endoscope includes a source of suction, the suction drawing food toward the tip, after which the device is removed from the esophagus.

14. A device for removing food from an esophagus of claim 13, the device comprising:
a flexible coupler;
the flexible coupler to connect to a flexible endoscope;
a tip;
the tip for interfacing with the food;
a fluid communication pathway connecting the flexible coupler to the tip;
a restriction within the fluid communication pathway, creating a venturi;
whereby the device is used in conjunction with the flexible endoscope to grasp food within the esophagus.

15. The device for removing food from an esophagus of claim 13, wherein:
the body is formed from a first chamber and a second chamber;
the first chamber having a first diameter and the second chamber having a second diameter;
the first diameter is equal to the second diameter.

16. The device for removing food from an esophagus of claim 15, wherein:
the first chamber has a first diameter and the second chamber has a second diameter; and
this first diameter is equal to the second diameter.

17. The device for removing food from an esophagus of claim 13, wherein:
the venturi includes a converging section, a diverging section, and a throat;
the converging section includes a venturi converging angle with respect to the throat;
the venturi converging angle being less than or equal to 15 degrees.

18. The device for removing food from an esophagus of claim 15, further comprising:
a flexible coupler;
the flexible coupler adjacent to the first chamber;
the flexible coupler allowing the device to be placed on and removed from a tip of an endoscope.

19. The device for removing food from an esophagus of claim 13, wherein:
the venturi is centered below a center of the body, creating a viewing recess;
the viewing recess allowing a user to have an unobstructed view through the body of the device.
